(19) United States
(12) Patent Application Publication
(10) Pub. No.: US 2011/0184306 A1
(43) Pub. Date: Jul. 28, 2011

(54) DEVICE FOR TREATING PARKINSON'S DISEASE AND METHODS OF USE THEREOF

(75) Inventors: Changqing Chris Kao, Brentwood, TN (US); Peter E. Konrad, Franklin, TN (US); Michael S. Remple, Nashville, TN (US); Joseph S. Neimat, Nashville, TN (US); P. David Charles, Nashville, TN (US)

(73) Assignee: VANDERBILT UNIVERSITY, Nashville, TN (US)

(21) Appl. No.: 13/011,906

(22) Filed: Jan. 23, 2011

Related U.S. Application Data
(60) Provisional application No. 61/299,322, filed on Jan. 28, 2010.
(60) Provisional application No. 61/299,196, filed on Jan. 28, 2010.

(51) Int. Cl. A61B 5/0476; A61N 1/36

(52) U.S. Cl. 600/544; 607/45; 600/300

(57) ABSTRACT

The invention includes a recordation and stimulation system for determining and delivering an electrical stimulation treatment based upon the current status of neuronal activity of a subject. The system include components for detecting neuronal activity in a subject's brain and, based upon the information received, determine an appropriate electrical stimulation treatment for the subject. The system allows immediate adjustments to the stimulation treatment as the needs of the subject change over time. The invention also includes a method for determining whether a subject has early Parkinson's Disease or advanced Parkinson's Disease. The method includes the steps of acquiring information regarding neuronal discharges in certain areas of the brain, creating a ratio based upon the neuronal activity, and determining whether a previous medical diagnosis of Parkinson's Disease is accurate.

This application claims the benefit of U.S. Provisional Patent Application Ser. No. 61/299,322, filed Jan. 28, 2010, entitled “Device for Treating Parkinson's Disease” which is hereby incorporated by reference in its entirety, and U.S. Provisional Patent Application Ser. No. 61/299,196, filed Jan. 28, 2010, entitled “Methods of Confirming a Medical Diagnosis of Parkinson's Disease” which is hereby incorporated by reference in its entirety.

Be it known that we, Changqing Chris Kao, a United States citizen, residing at 554 Lester Court, Brentwood, Tenn. 37027, Peter E. Konrad, a United States citizen, residing at 3013 Boxwood Drive, Franklin, Tenn. 37069, Michael S. Remple, a Canadian citizen, residing at 804 North Woodstone Lane, Nashville, Tenn. 37211, Joseph S. Neimat, a United States citizen, residing at 213 Carden Avenue, Nashville, Tenn. 37205, P. David Charles, a United States citizen, residing at 6509 Edinburgh Drive, Nashville, Tenn. 37221, have invented a new and useful “Device for Treating Parkinson's Disease and Methods of Use Thereof.”

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

Parkinson's disease (PD) is a progressive and disabling neurodegenerative disorder affecting over one million people. The current standard of care, dopamine replacement with levodopa, improves the symptoms but to date, no pharmaceutical, biologic, procedure, or device has been proven to slow the relentless progression. Increasingly higher doses of anti-PD medications are needed for adequate symptom control, and the risk of developing motor complications of therapy is 50-75% within seven years of initiation.

Deep brain stimulation (DBS) surgery is effective for treating certain medical conditions. DBS of subthalamic nucleus (STN) treats the symptoms of Parkinson's disease by electrical stimulation. The efficacies are target dependent. Currently available hardware delivers the electrical stimulation based upon the settings provided by the clinician. That is, implantable stimulation devices for movement disorders are output only. Accordingly, the electrical stimulation settings of a device remain constant until the settings are modified for some reason at a point in the future. What is needed is a more responsive way to alter the electrical stimulation settings based upon the patient's immediate needs.

Even without considering this possible influence on clinical progression, applying DBS in earlier stages of PD than is currently practiced holds promise because the therapy may provide better symptom control and quality of life than medications alone. Furthermore, after implantation, patients with DBS require less medication. Accordingly, what is needed is a method to distinguish patients having earlier stages of PD from patients having advanced PD. DBS improves the quality of life of the patient, and optimizing the stimulation settings improves the quality of DBS therapy.

### SUMMARY OF INVENTION

The present invention provides a recordation and stimulation system for determining neuronal activity levels in a subject's brain and then using that information to determine an appropriate electrical stimulation treatment. That is, subjects having Parkinson's Disease respond to electrical stimulation treatment of the brain. Optimization of that electrical stimulation treatment will remove the negative effects of over-stimulation. Currently, implantable stimulation devices for movement disorders are output only. Accordingly, there is a need for the current invention. The present invention is a device that includes an electronic switch in order to allow input and output within the same second. That is, during one fraction of a second, the electronic switch allows input to the device of neuronal activity levels in the specific areas of a subject's brain. Then, during the next fraction of that second, the device outputs a specific electrical stimulation treatment based upon the information just received in the previous fraction of a second. Accordingly, the present invention provides for inputting information about a subject and outputting electrical stimulation to that subject's brain so that the treatment scheme very nearly matches the needs of the subject at that point in time. In certain embodiments, a device for providing electrical stimulation, includes, a housing, an electronic switch, an amplifier attached to the electronic switch, a convertor attached to the amplifier, a microprocessor attached to the convertor and the housing, an integrator attached to the microprocessor, and a stimulator attached to the integrator and the electronic switch. In other embodiments, the device further includes a scanner operationally connected to the microprocessor. In still other embodiments, the device further includes a lead and a cable attached to the electronic switch.

In still other embodiments, the invention is a recordation and stimulation system, including a lead, a cable operationally connected to the lead, a housing, an electronic switch operationally connected to the cable, an amplifier operationally connected to the electronic switch, an analog to digital convertor operationally connected to the amplifier, wherein the convertor is attached to the housing, a microprocessor operationally connected to the convertor, wherein the microprocessor is attached to the housing, an integrator operationally connected to the microprocessor, and a stimulator operationally connected to the integrator and the electronic switch, wherein the stimulator is attached to the housing. In still other embodiments, the recordation and stimulation system further includes a scanner operationally connected to the microprocessor. In yet other embodiments, the invention is an electronic stimulation device as shown and described herein. In still other embodiments, the invention is a device comprising a recordation and stimulation system as shown and described herein. In still other embodiments, the invention is a method of using a device comprising the steps as shown and described herein. In alternate embodiments, the invention is a method of manufacturing a recordation and stimulation system as shown and described herein.

The present invention also provides a method for confirming a medical diagnosis of Parkinson's Disease. The invention discloses the steps of obtaining neuronal activity of the subthalamic nucleus (S TN) and the substantia nigra (SN) and the steps of determining an STN/SN ratio in order to determine whether a previous medical diagnosis of Parkinson's Disease for a subject is accurate. Due to similarities of the clinical symptoms of Parkinson's Disease to other diseases or conditions, Parkinson's Disease has a misdiagnosis rate of up to 35%. Currently, verification of a diagnosis of Parkinson's Disease is by a post-mortem histological confirmation, in which neurons are counted to determine neuronal loss. The present invention detects neuronal activity with electrophysiology. In certain embodiments, the invention is a method of calculating a subthalamic nucleus to substantia nigra ratio, including measuring activity of a subthalamic nucleus, measuring activity of a substantia nigra, calculating a ratio of the subthalamic nucleus activity to the substantia nigra activity, and confirming a medical diagnosis of Parkinson's Disease based upon the ratio. In yet other embodiments of the invention, confirming a medical diagnosis further includes diagnosing Parkinson's Disease by comparing the ratio to the ratio of a subject with early Parkinson's Disease (Hoehn and Yahr stage II) or advanced Parkinson's Disease. In other embodiments of the invention, the invention is a method of obtaining a subthalamic nucleus to subtantia nigra ratio, including inserting a lead into a brain, recording a neuronal activity of an area of a subthalamic nucleus, recording a neuronal activity of an area of a substantia nigra, determining a ratio of the subthalamic nucleus neuronal activity to the substantia nigra neuronal activity, and displaying the ratio as a visual display. In other embodiments, the invention further includes comparing the ratio to at least one reference ratio including a ratio of neuronal activity in the subthalamic nucleus to neuronal activity in the substantia nigra for a subject having advanced Parkinson's Disease. In still other embodiments, the invention is a method of using an implantable lead for diagnosing or confirming a diagnosis of Parkinson's Disease in a subject in which the lead is implanted, the method including tracking neuronal activity in a subthalamic nucleus, tracking neurnal activity in a substantia nigra, determining a ratio of neuronal activity in the subthalamic nucleus to neuronal activity in the substantia nigra, and comparing the ratio to at least one reference ratio including a ratio of neuronal activity in the subthalamic nucleus to neuronal activity in the substantia nigra for a subject having advanced Parkinson's Disease. In still other embodiments, the invention includes a method of determining the status of a Parkinson's Disease subject, including inserting a first lead into a brain, inserting a second lead into a brain, recording a neuronal activity of a subthalamic nucleus, recording a neruonal activity of a substantia nigra, determining a ratio of the neuronal activity of the subthalamic nucleus to the neuronal activity of the substantia nigra, displaying the ratio as a visual display, and diagnosing Parkinson's Disease based upon the visual display. In still other embodiments of the invention, the invention includes determining the ratio by using a volts meter. In yet other embodiments of the invention, the invention further includes diagnosing Parkinson's Disease by comparing the ratio to a known ratio of a subject with early Parkinson's Disease (Hoehn and Yahr stage II) or advanced Parkinson's Disease. In still other certain embodiments, the invention includes a method of determining a ratio of an activity of a subthalamic nucleus of a subject's brain to an activity in a substantia nigra of the subject's brain including the steps shown and described herein. In still other embodiments, the invention is a method of using a ratio of an activity in a subthalamic nucleus of a subject's brain to an activity in a substantia nigra of the subject's brain including the steps as shown and described herein. In still other embodiments, the invention is a method of confirming a diagnosis of Parkinson's Disease as shown and described herein. In yet other embodiments, the invention includes the methods as shown and described herein.

Accordingly, one provision of the present invention is to provide a method of determining whether a subject has Parkinson's Disease.

Still another provision of the present invention is to provide a method of using the ratio of neuronal activities of the subthalamic nucleus to the substantia nigra in order to determine whether medical diagnosis of Parkinson's Disease is correct.

Another provision of the present invention is to provide a method of determining an STN/SN ratio of a subject for comparison of that ratio to STN/SN ratios of other subjects having either early Parkinson's Disease or advanced Parkinson's Disease.

Yet another provision of the present invention is to provide a device for determining the status of a Parkinson's Disease subject so that an appropriate electrical stimulation treatment may be determined and delivered based upon the status of the subject.

Still another provision of the present invention is to provide a device for determining neuronal activity levels, calculating a ratio of those activity levels, and determining an appropriate electrical stimulation treatment based upon the neuronal activity levels.

Another provision of the present invention is to provide a closed loop recordation and stimulation system for determining stimulation treatment based upon subject status input into the device.

Still another provision of the present invention is to provide brain stimulation treatment which is determined by and dependent upon the current status of the Parkinson's Disease of the subject.

### BRIEF DESCRIPTION OF THE DRAWINGS

FIG. 1 is a schematic diagram of an embodiment of the present invention. Shown therein are the elements and operational connections of the recordation and stimulation system disclosed herein.

FIG. 2 is a flow diagram of an embodiment of the present invention. The flow chart illustrates the manner of detecting neuronal activity, determining an appropriate electrical stimulation treatment for output, and delivering the appropriate electrical stimulation treatment to the subthalamic nucleus.

FIG. 3 is a table showing the characteristics of electrical stimulation to be delivered dependent upon the STN/SN ratio determined by the neuronal activity detected by the present invention. Accordingly, the table may be used to determine the appropriate voltage, frequency and duration of the electric stimulation to be delivered.

FIG. 4 is a schematic diagram of contacts and wires within a lead showing how electric signals are carried to the contacts for delivery to the neurons surrounding them.

FIG. 5 is a schematic diagram of another embodiment of a lead in which detecting electrodes are mounted on the lead in order to receive neuronal activity input from neurons surrounding them. Note that the ordinary wiring content of the lead (best seen in FIG. 4) is not shown in this figure.

FIG. 6 is a schematic diagram of another embodiment of contacts and wires within a lead showing how electric signals are carried to the contacts for delivery to the neurons surrounding them.

FIG. 7 is a flowchart showing an embodiment of the method of obtaining neuronal activity within the subthalamic nucleus (STN) and the substantia nigra (SN) and using that information to confirm a medical diagnosis of Parkinson's Disease.

FIG. 8 is a schematic drawing of a recording of a neuronal discharge. Shown there is the amplitude and duration, which are characteristics used to calculate the pRMS, as further disclosed herein.

FIG. 9 is a schematic drawing showing a side view of a human brain. Shown therein is the approximate location within the brain of the subthalamic nucleus and the substantia nigra. Also shown is a lead which is inserted into the brain in order to record neuronal activity in the specific sections shown, as further described herein.

FIG. 10 is a schematic drawing showing a rear view of a human brain. Shown there is the simultaneous bilateral procedure in which a lead is placed in each hemisphere of the brain at an identical position and depth relative to the hemisphere.

FIG. 11 is a table displaying the L-Dopamine response, UPDRS values and STN/SN ratios of a population of subjects having early Parkinson's Disease. Information for each of the nine subjects is shown.

FIG. 12 is a table displaying the L-Dopamine response, UPDRS values and STN/SN ratios of a population of subjects having advanced Parkinson's Disease. Information for each of the nine subjects is shown.

FIG. 13 is a bar graph showing the ratios of the pRMS of the neuronal activity within subthalamic nucleus to the neuronal activity within the substantia nigra within the left hemisphere of the brain of subjects having early or advanced Parkinson's Disease. For each listed age, the bar graph on the left indicates the STN/SN ratio for the early Parkinson's Disease subject and the bar graph on the right indicates the STN/SN ratio for the advanced Parkinson's Disease subject.

FIG. 14 is a bar graph showing the ratios of the pRMS of the neuronal activity within subthalamic nucleus to the neuronal activity within the substantia nigra within the right hemisphere of the brain of subjects having early or advanced Parkinson's Disease. For each listed age, the bar graph on the left indicates the STN/SN ratio for the early Parkinson's Disease subject and the bar graph on the right indicates the STN/SN ratio for the advanced Parkinson's Disease subject.

### DESCRIPTION OF THE PREFERRED EMBODIMENTS

The present invention is a device 10 for providing electrical stimulation to a subject's brain based upon neuronal activity sensed by the device 10. In certain embodiments, the device 10 includes an amplifier 14, electronic switch 16, lead 18, and cables 20. In other embodiments, the device 10 includes a lead 18, cables 20, electronic switch 16, amplifier 14, analog to digital converter 24, microprocessor 28, power source 31, integrator 29, and stimulator 12. Electrical stimulation of the brain is a preferred therapeutic technique for several diseases, including Parkinson's Disease. Deep brain stimulation (DBS) is a procedure in which an implantable lead 18 is placed at a specific location within the brain 22 in order to provide specific electrical stimulation to that particular area of the brain 22. Alteration of the stimulation treatment automatically based upon neuronal activity at specific locations within the brain 22 is highly desired. Optimal treatment by such electrical stimulation is achieved by varying the treatment instantly based upon the information from the brain 18 which is being stimulated.

DBS procedures include the steps of implanting a lead 18 in a brain 22 with the subsequent electrical stimulation of the elements, or contacts, of that lead 18 by a generator. The generator is attached to the lead 18 by a cable 20. The electrical stimulation perimeters provided have three components. Those components are voltage, frequency (expressed as Hertz), and pulse duration. In the case of Parkinson's Disease, a subject's need for electrical stimulation varies as that subject is awake, asleep, exercising vigorously, or relaxed. At least one of the points of novelty about the invention disclosed herein is that the device 10 is capable of delivering electrical stimulation based upon neuronal activity information received by the device 10.

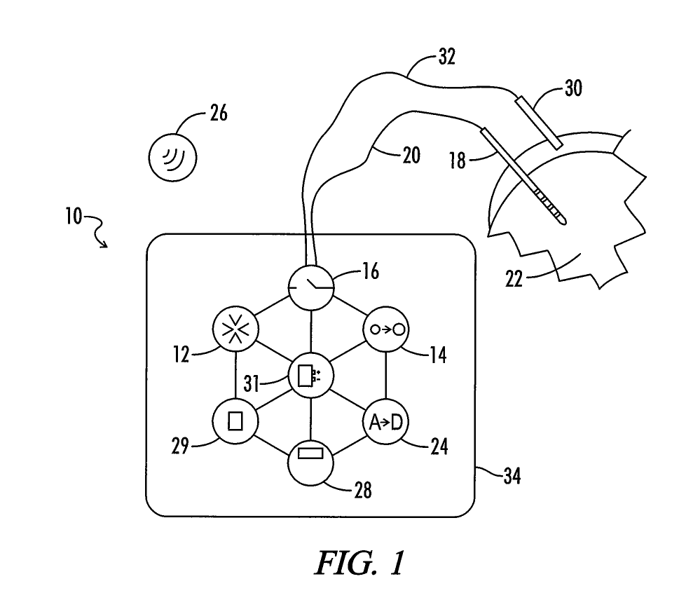

Referring now to FIG. 1, there is shown an embodiment of the present invention. The device 10 include a stimulator 12, which provides the electrical stimulation being output by the device 10. An example of a suitable stimulator 12 is a model called Synergy Dural channel Itrel, which is commercially available from Medtronic, Inc., of Minneapolis, Minn. 55423-5604. The output from the stimulator 12 goes through the cable 20 to the lead 18 so that the electrical stimulation is delivered through the lead 18 to the brain 22. An example of a suitable cable 20 is model number 7482—Extension, commercial available from Medtronic, Inc., of Minneapolis, Minn. 55423-5604. An example of a suitable lead 18 is any standard DBS implantable lead. An example of such a lead is model 3389, which is commercial available from Medtronic, Inc., of Minneapolis, Minn. 55423-5604 In addition to outputting an electrical stimulation, the device 10 is capable of receiving electrical input. By way of example, the device 10 may receive input such as a measure of neuronal activity from an area of the brain 22 that is adjacent to the lead 18. Such procedures are further described herein. Neuronal activity is captured by the elements of the lead 18. The signal travels from the lead 18 through the cable 20 to device 10. The input signal is amplified by an amplifier 14. An example of an amplifier 14 is a differential DC amplifier with input impedance balanced at 200 Mohm (mega ohm), noise level at 0.6 μV (2 Hz-10 kHz.0 sensitivity at 0.5 μV/D-20 mV/D (volts/division). By way of example, an example of an amplifier 14 is model named Leadpoint, which is commercially available from Medtronic, Inc., of Minneapolis, Minn. 55423-5604.

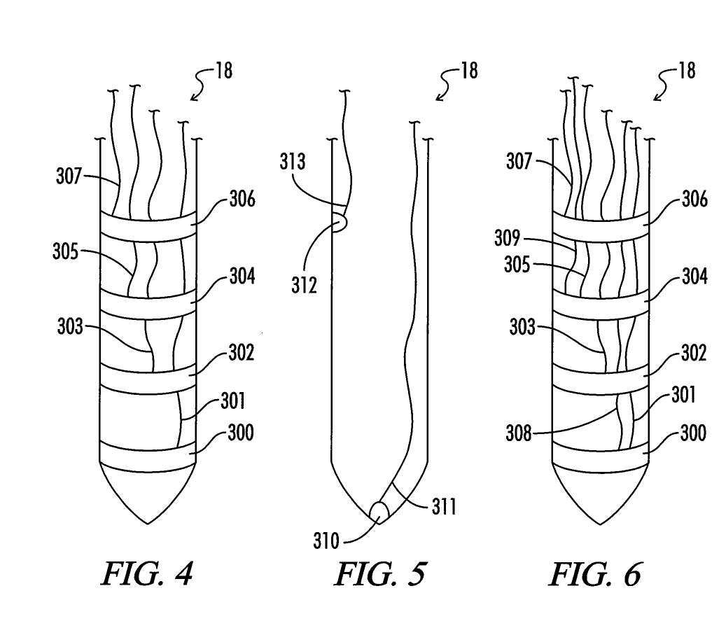

Currently, commercially available leads 18 are used for stimulation treatment only. That is, they are not used for receiving input from the brain 22. In a first embodiment, as best seen in FIG. 4, a modification to the lead 18 results in the ability of the lead 18 to receive electrical signals regarding neuronal discharges sensed by the contacts of the lead 18, as well as outputting electrical stimulation. As shown in FIG. 4, a lead 18 has four contacts which are for delivering electrical stimulation, such as during electrical stimulation treatment. Each contact has its own wire. That is, the first contact 300 outputs electricity received through the first connector 301. The second contact 302 uses the second connector 303. The third contact 304 uses the third connector 305. The fourth contact 306 uses the fourth connector 307. Such a lead 18 is model number 3389, commercially available from Medtronic, Inc., of Minneapolis, Minn. However, in order to detect neuronal activity, the current lead needs to be modified. After modification as disclosed herein, each of the connectors 301, 303, 305 and 307 is capable of both outputting electrical stimulation and receiving input from the brain 22. In another embodiment, as best seen in FIG. 6, the contact (macro tip (1.27×2 mm) 300 has an extra connector 308 for detecting activity with in the SN and the third contact 304 has an extra connector 309 for detecting neuronal activity in the STN. The other alternate embodiments, there may be two sets of smaller leads, each having detecting electrodes, micro tip (30-50 μm), semi-macro tip (0.3-0.5 mm) and serving the same function in detecting neuronal activities. They are mounted next to the contacts as described and shown below. In certain embodiments, stimulation treatment may be directed only to the STN, and not the SN. In other embodiments, the device 10 disclosed herein provides the user the option to determine which of the four contacts to provide stimulation to.

In a second embodiment, as best seen in FIG. 5, a first detecting/recording electrode 310 is mounted at the very tip of the lead 18 to detect SN neuronal activity. Note that for the model listed above there is a 1.5 mm×1.27 mm rounded plastic tip at one end and the detecting tip of the electrode 310 is located at this location. A second detecting electrode 312 tip is located 3.25 mm above the first electrode 310 so that the second detecting electrode 312 detects STN activity. The first electrode connector 311, and the second electrode connector 313, which are connected to the respective electrodes, are shielded and bundled with other wires within the lead 18 and then connected to the electronic switch 16. Note that FIG. 5 does not show the contacts and connectors which are shown in FIG. 4. The tip of each electrode 310, 312 is 100% platinum. The platinum is connected to platinum-iridium wire insulated by plastic, or the like (such as polythurethan film). The first electrode connector 311, and the second electrode connector 313 are shielded by copper mash cable in addition to the insulation. The total diameter of the cable is about 1.5 mm. As seen in FIG. 5, in aggregate, there are six connectors, such that there are four outputs for stimulation (while the switch allows stimulation, which, in certain embodiments, is 80% of the time during a second—that is allowing stimulation for a period of 800 ms), and two inputs to the device 10 to detect neuronal activities (the switch allowing receipt of such signal for 20% of each time period—that is 200 ms).

Referring back to FIG. 1, a first lead 18 is attached to a cable 20, which is then attached to an electronic switch 16. Also shown is a second lead 30 attached to a second cable 32 which is attached to the same electronic switch 16. One method of modifying currently available cables is provided above. Other cables capable of transferring the signal, as disclosed herein, are commercially available. In the embodiment shown, the electronic switch 16 is used to dictate the direction of information flow, as further described elsewhere in this application.

Referring to the housing 34 of the device 10, the housing 34 may be any suitable lightweight, biocompatible material such as, an appropriate plastic, rubber, or metal, or the like. The device may be implanted into the subject's body. Methods of manufacturing and shaping materials suitable for the housing 34 are known to those skilled in the art and such services are readily commercially available. In certain embodiments of the present invention, the size of the device 10 is generally small, for convenient implantation into a subject's body. In other embodiments of the present invention, the device 10 may have an alternate shape, such as a square, rectangle, or the like.

The present invention may use various power sources and power supplies as described herein, or known to those of ordinary skill in the arts. In certain embodiments, the energy source 31 is attached to, and provides a power source for, the elements disclosed herein needing power for operation, as known to those of ordinary skill in the art. In certain embodiments, the energy source 31 may be a battery, or the like. Such batteries are known in the art and are readily commercially available. In certain embodiments of the present invention, the energy source 31 is removable battery. In other embodiments, the energy source 31 may be any energy source known by those of ordinary skill in the art which would provide sufficient power to the other elements for their operation in the manner described herein. In still other embodiments, the energy source 31 is a non-removable battery. In certain embodiments, the invention may include a resistor in order to match the electrical capabilities of the energy source 31 with the output ability of the other elements described herein. The present invention includes proper electrical insulation, as known by those skilled in the art, so that a subject is not shocked and so that proper function occurs under the use circumstances described herein. In certain embodiments, the energy source 31 is operationally connected to the electronic switch 16, amplifier 14, converter 24, microprocessor 28, integrator 29, and stimulator 12.

Still referring to FIG. 1, note that the connections between the components of the present invention are those operational connections known to those of ordinary skill in the arts. The schematic diagram uses lines to demonstrate the operational connectivity of the parts shown (i.e., wires, or other means, attaching, or allowing communication, or connectivity, so that, for example, the microprocessor 28 signal to the stimulator 12, through the integrator 29, results in an appropriate electrical stimulation output). Operational connectivity includes any connections necessary for power, data or information transfer, or the like, for the operation of the specific device. One of ordinary skill in the art is familiar with such types of connections. For example, as seen in FIG. 1, the analog to digital converter 24 is operationally connected to the microprocessor 28. As a second example, the stimulator 12 is operationally connected to the electronic switch 16. Applicant discloses herein various embodiments of the device 10 to function as described herein.

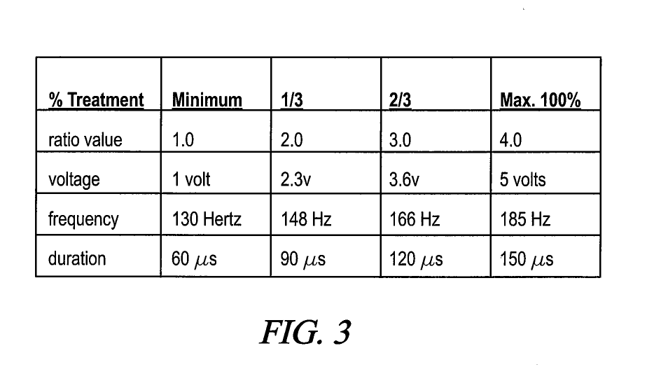

In certain embodiments of the invention, the electronic switch 16 allows input into the device 10, and the input is directed to the amplifier 14, as described above. After amplification, the analog signal passes to an analog to digital convertor 24. Such convertors 24 are well known in the art and widely commercially available. The convertor 24 is operationally connected to a microprocessor 28. The microprocessor 28 may be a computer, controller, microprocessor, or processor that can receive the detected neuronal activity, calculate pRMS, determine the STN/SN ratio, determine the electrical treatment to be output, and compare that to known settings, as further disclosed herein. Such microprocessors 28 are known to those of skill in the art and are readily commercially available, for example from Texas Instruments Incorporated of Dallas, Tex. or Dell, Inc. of Round Rock, Tex. The microprocessor 28 operationally connects to an integrator 29. Integrators 29 are well know in the art and widely commercially available. The integrator 29 operationally connects to a stimulator 12. Stimulators 12 are well known in the art and commercially available from the sources indicated herein. The electrical output, which may have the characteristics as further described in FIG. 3, pass through the electronic switch 16, according to the settings of the electronic switch 16. The output stimulation reaches the destination, as further described herein, as it passes through the cables 20, 32 to the leads 18, 30 for delivery to the brain 22.

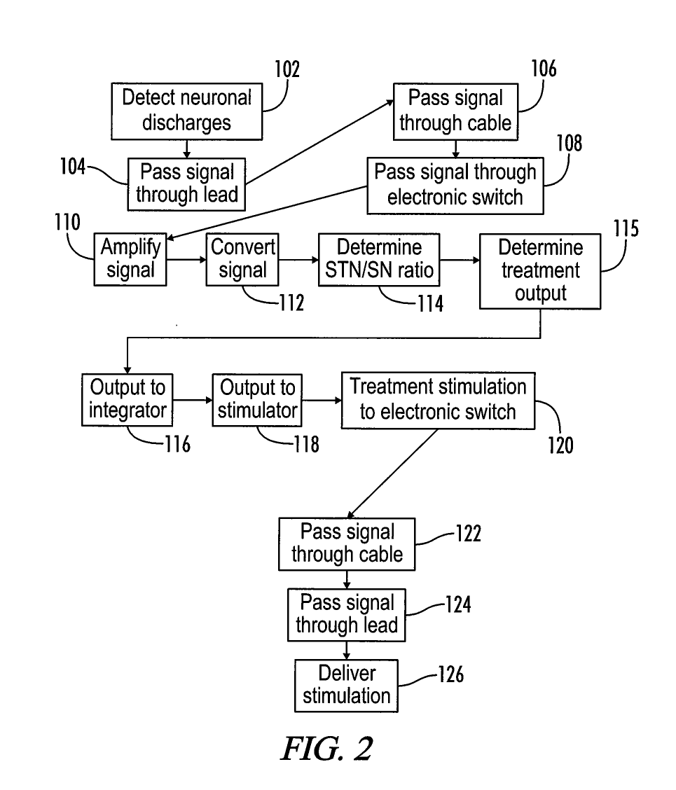

Referring now to FIG. 2, there is shown a flow diagram of an embodiment of the invention. The device 10 detects neuronal discharges 102 through the context of a lead 18. The analog signal passes through a lead 104 and passes through a cable 106 to reach an electronic switch 16. When the electronic switch 16 allows input to the device 10, the analog signal passes through the electronic switch 108 and is amplified by an amplifier 110. The analog signal is then converted to a digital signal as it passes through an analog to digital convertor 112 to the microprocessor 28. The microprocessor 28 receives the digital signal, determines the pRMS, as further described elsewhere herein, and determines the STN/SN ratio 114, as further described herein. The microprocessor 28 compares the STN/SN ratio to the treatment coordination chart, shown in FIG. 3, in order to determine the characteristics of the electrical stimulation to be output by the device 10. After determining the voltage, frequency, and duration of the electrical stimulation to be output, those characteristics of the output signal passes to the integrator 116. Integration of the specific characteristics of the output signal then pass to the stimulator 118 so that the desired electrical stimulation is generated by the stimulator 12. The output stimulation passes through the electronic switch 120, as permitted, according to the settings of the electronic switch 16. The output signal passes through cables 122 to reach the leads 124 in order to deliver stimulation 126 to the desired location of the STN.

In other embodiments, the device 10 includes elements for recording the input signal, manipulating the input signal by mathematical calculation, comparing the manipulated information to set standards, and altering the electrical output from the device 10 based upon the same. By way of example, with regard to Parkinson's Disease, neuronal activity of two specific areas of the brain, as further described elsewhere herein, may be sensed by the lead 18. Such electrical input reaches the amplifier 14 and is amplified before being recorded by the microprocessor 28. As further described elsewhere herein, the calculation of the peak root mean square (pRMS) of the neuronal activity is calculated. The microprocessor 28 records the input signal, calculates the pRMS of that value and records the same. Note that a standard lead 18 has multiple (typically 4) elements which may be used for outputting electrical signal or receiving an input signal. Accordingly, multiple input signals may be received, recorded, and manipulated at the same time. With regard to Parkinson's Disease, one such input signal may be the neuronal activity of the STN and another input could be the neuronal activity of SN. Accordingly, the microprocessor 28, after calculating the pRMS of each, determines a ratio in which the pRMS of STN is the numerator and the pRMS of SN is the denominator. This STN/SN ratio may be referred to as the ratio. The microprocessor 28 then compares the ratio to known standards (see FIG. 3) in order to deduce the voltage, frequency, and duration of the electrical stimulation which the device 10 will output to the lead 18.

Referring now to FIG. 3, there is shown a table that may be used to deduce the voltage, frequency and duration of the electrical stimulation being provided by the device 10. Accordingly, the processor 28 uses the ratio to determine the appropriate voltage, frequency and duration of the output electrical signal. The row labeled “ratio value” refers to the STN/SN ratio, as further described below. Based upon what the STN/SN ratio value is determined to be, the characteristics of the stimulation treatment vary according to the table. That it, in addition to selecting the numbers shown, the table should be considered a spectrum from which treatment characteristics may be selected based upon the exact ratio value. For example, an STN/SN ratio of 2.0 would result in an electrical signal output having the characteristics of 2.3 volts, 148 hertz (Hz) for a duration of 90 microseconds. In certain embodiments, stimulation treatment is delivered only to the STN, and not the SN.

The device 10 includes an electronic switch 16 that allows the device 10 to switch between the functions of outputting an electrical signal and receiving an input signal. In certain embodiments, the electronic switch 16 may oscillate between output/input based upon a remote switch which dictates the same. In other embodiments, the electronic switch 16 may be an automatic switch in which the device 10 is providing an output electrical signal 80% of the time and receiving an input signal 20% of the time during each one second period of time. In still other embodiments, the electronic switch 16 may be programmed to specific output/input characteristics. Such electronic switches 16 are well known in the art and readily commercially available.

The device 10 described herein is implantable within the human body. Uses of the invention include monitoring the progression of the disease and providing treatment as described herein. For example, regarding Parkinson's Disease, the ability of the device 10 to receive an input signal regarding the activity of the STN and SN, allows the progress of the disease to be monitored based upon the ratio, as set forth herein. Further, this device may be used to explain unresponsive subjects who were inaccurately diagnosed with Parkinson's Disease. That is, there are several other diseases which mimic the clinical symptoms of Parkinson's Disease. Accordingly, if a subject is unresponsive to DBS, the information received from this device 10 will allow for a confirmation of the medical diagnosis.

In certain embodiments, a scanner 26 is operationally connected to the microprocessor 28 in order to reprogram or reset the characteristics of the microprocessor 28, or other elements of the device 10 as shown in FIG. 1, and described herein. The scanner 26 is located outside of the subject's body. Such scanners 26 are well known in the art. Examples of commercially available scanners 26 include model nos. 7432, 7433, and N/Vision 8840AAI from Medtronic, Inc. Thus, the operational connection between the scanner 26 and the microprocessor 28 is a wireless communication, or the like.

One of the problems solved by the current device 10 is a problem of unnecessary electrical stimulation of the brain. Electrical overstimulation of the brain results in the release of glutamate which kills neurons. Various physical circumstances of a subject result in the need for various electrical stimulation of the brain during those circumstances. The closed loop device 10 disclosed herein provides for such therapy.

The present invention relates to a method of obtaining and using a ratio of the neurological activity within the subthalamic nucleus (STN) of a subject's brain 22 to the neurological activity within the substantia nigra pars compacta (SNc). Currently, Parkinson's Disease has no known cause and may only be verified by a post-mortem histological confirmation. Parkinson's Disease has a misdiagnosis rate of up to 35%. Proper and efficient treatment for Parkinson's Disease results when the subject is actually suffering from Parkinson's Disease, rather than some other physiological condition which mimics several of the Parkinson's symptoms. Briefly, treatment for Parkinson's Disease may include electrical stimulation of certain areas of the brain 22. Excessive, or unnecessary, stimulation is not optimal treatment. Progressive SNc dopaminergic neuron loss in Parkinson's Disease may indirectly cause STN cell hyperactivity and consequent SNc excitotoxic damage. That is, glutamate toxicity is a result for the SNc neuronal loss. Idiopathic Parkinson's Disease results when 75% of neuronal function in the substantia nigra is lost. Given the high rate of misdiagnosis of Parkinson's Disease and the several other conditions which may not result in neuronal loss in the SNc for some reason, the invention disclosed herein is important for the proper diagnosis of Idiopathic, or primary, Parkinson's Disease.

The invention includes a method of calculating a STN/SN ratio. Herein, the STN/SN ratio may be referred to as the “ratio.” At least one of the novel aspects of the invention is the calculation of the STN/SN ratio in combination with the subsequent use of that ratio for the purpose of confirming a medical diagnosis of Parkinson's Disease. Given the medical problems of misdiagnosis of this disease, there is a need for a method of confirming a medical diagnosis so that subjects not having Parkinson's Disease, and merely displaying symptoms similar to several Parkinson's symptoms, may be identified so that they do not incur the time, expense, and emotion of undergoing a deep brain stimulation procedure in an attempt to treat the Parkinson's Disease.

In certain embodiments, the ratio may be presented as a visual display. Visual displays of the ratio may include bar graphs, pie graphs or other visual depictions indicative of a status of Parkinson's Disease. In other embodiments, the visual display of the ratios may take the form of a confirmation of a medical diagnosis of Parkinson's Disease, or some other indication of an affirmative result relevant to an existing medical diagnosis.

In certain embodiment of the invention, the method of obtaining a STN/SN ratio includes the initial steps of inserting a first lead 18 into the brain 22, as seen in FIG. 3. In certain embodiments, a bi-lateral procedure allows insertion of a lead 18 into each hemisphere of the brain 22 so that a ratio for each hemisphere of the brain 22 results, as seen in FIG. 4. After insertion of a lead 18, there is recording of the activity of the subthalamic nucleus, recording an activity of the substantia nigra, determining the pRMS of the values, as further described below, calculating a subthalamic nucleus/substantia nigra ratio, displaying the ratio as a visual display, and confirming a medical diagnosis of Parkinson's Disease based on the visual display. In addition to the high rate of misdiagnosis for Parkinson's Disease, the deficiency of a deep brain stimulation procedure for the implantation of a deep brain stimulation (DBS) implantable lead depends on targeting and delivering to a precise location. Such implantation procedures are very expensive.

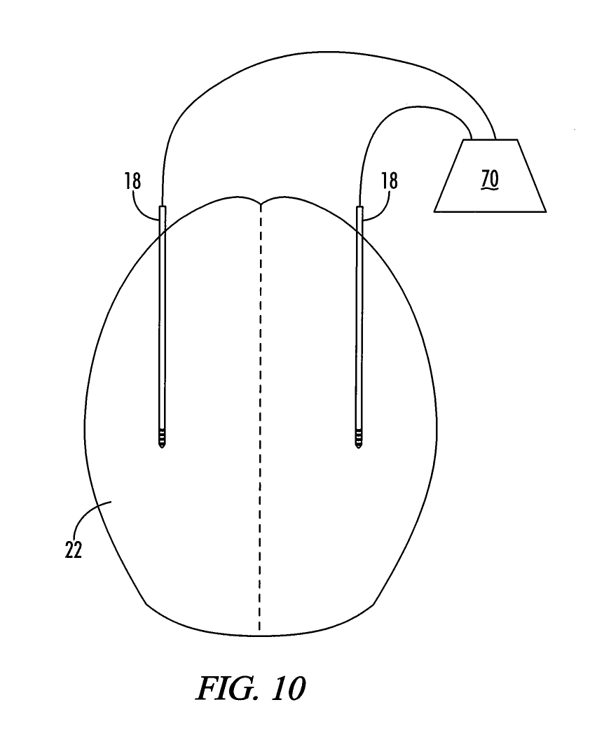

In other embodiments of the invention, the method of obtaining a STN/SN ratio includes the initial steps of inserting a first lead 18 into the brain 22, as seen in FIG. 9. In certain embodiments, a bi-lateral procedure allows insertion of a lead 18 into each hemisphere of the brain 22 so that a ratio for each hemisphere of the brain 22 results, as seen in FIG. 10. After insertion of a lead 18, there is recording of the activity of the subthalamic nucleus, recording an activity of the substantia nigra, determining the pRMS of the values, as further described below, calculating a subthalamic nucleus/substantia nigra ratio, and determining treatment.

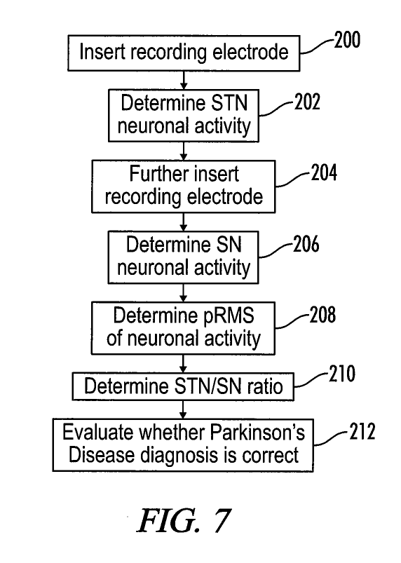

Referring now to FIG. 7, there is shown a flow chart of the steps of the method disclosed herein. Briefly, detection of neuronal activity in the STN and SN results from inserting a lead 18, or a plurality of leads 18, into a brain 22, as further described herein. As best seen in FIG. 7, in certain embodiments, the method includes inserting a recording electrode 200 (commercially available from FHC, Inc., of Bowdoin, Me.) to a position within a brain 22 to determine STN neuronal activity 202. In certain embodiments, neuronal activity is measured in mV. Further inserting the recording electrode 204 to another position within the brain 22, such as the SN, will determine the SN neuronal activity 206. In certain embodiments, as described elsewhere herein, the increments of movement within the brain 22 occur at 0.5 mm intervals. The next step in the procedure is to determine the pRMS of each neuronal activity recordation 208. After having neuronal activity for the STN and SN, and the pRMS for each, the next step is to determine the STN/SN ratio 210. In certain embodiments, the ratio is then compared to FIG. 3 to determine appropriate stimulation treatment. In other embodiments, by comparing the ratios to the actual ratios indicative of early Parkinson's Disease or advanced Parkinson's Disease, as disclosed herein, the next step is to evaluate whether the Parkinson's Disease diagnosis is correct 212. By way of background, note that the lead 18 may be the final implant, after the recording electrode has been used.

The STN activity and SN activity may be obtained by inserting a lead 18, as during a deep brain stimulation (DBS) procedure. Details of such a procedure are disclosed elsewhere in this application. An example of a suitable implantable lead 18 is the Medtronic 3389 or 3387 DBS lead, commercially available from Medtronic, Inc. Also, St Jude's of Memphis, Tenn., has a DBS lead called Libra, which is commercially available. When the lead 18 is present in the STN or SN, in either hemisphere of the brain, the activity of the neurons is recorded as electronic waves. As further described below, the peak root mean square (pRMS) of the activity is then computed.

By way of background regarding the detection of neuronal activity in a subject's brain 22, there are at least three ways to obtain recordings of neuronal activity. The various ways include microelectrode recording, DBS lead recording, and semi-macro recording. Briefly, microelectrode recordings (MER) require insertion of microelectrodes such that activity of the neurons surrounding the tips of electrodes from various parts of the brain 22 is detected. Second, DBS lead 18 recording is a procedure in which an implantable DBS lead 18 is inserted after microelectrode recording has occurred. The DBS lead 18 is then positioned such that the multiple electrodes on the lead 18 are positioned within the STN and SN so that the neural activity of those two areas is recorded. Third, semi-macro recording is the recordation of a neural activity pool bigger than MER, and smaller than the DBS lead technique. Semi-macro recording occurs during the MER procedure, described above, wherein the tip of the cannula is the MER electrode. In this situation, the cannula is connected to the recordation system 70 in order to record neuronal activity. Each of these procedures is well known in the art and generally known to those of ordinary skill in the art. Materials and equipment to perform each of these procedures is readily commercially available.

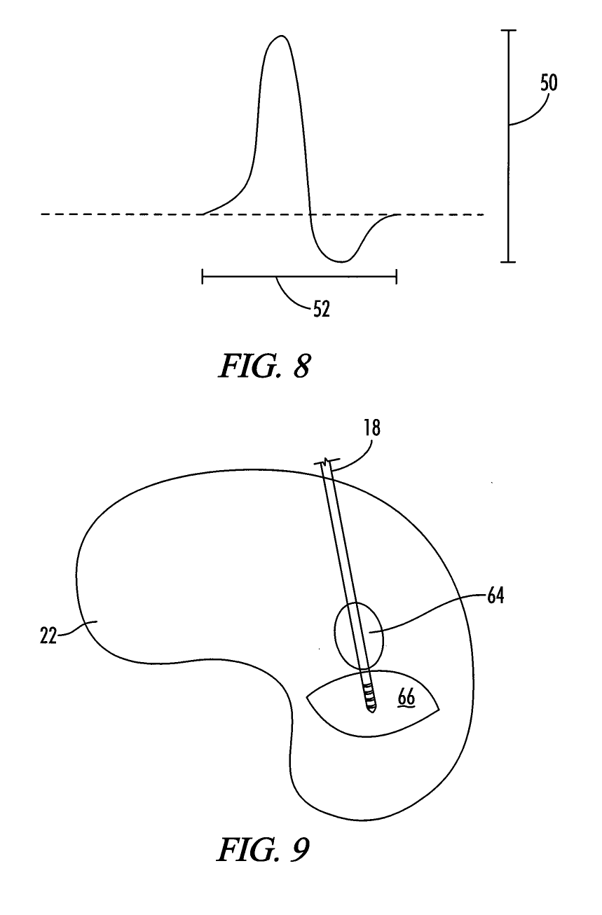

Referring to FIG. 8, there is shown a schematic drawing of a wave of neuronal activity. By way of background, note that each wave has an amplitude 50, duration 52 and frequency. As further described in Example 1, in certain embodiments, recordations of neuronal activity lasting ten seconds may be used for the calculation of pRMS. In still other embodiments, recordations may be 15 or 20 seconds in duration. In other embodiments, recordations may be only a fraction of a second, depending upon the settings of the electronic switch 16. The following is an example of a method of determining the STN/SN ratio. Within a ten second trace, the peak value is identified. That is, the single highest peak in that duration is identified. It is the peak root mean square that is subsequently used to determine the STN/SN ratio. After waves of neuronal activity have been recorded for some length of time, that information is used to compute a peak root mean square (pRMS). By way of background, the root mean square (RMS) value of a set of values (or a continuous-time waveform) is the square root of the arithmetic mean (average) of the squares of the original values (or the square of the function that defines the continuous waveform) (Watkins P. T., Santhanam G., Shenoy K. V. and Harrison R. R., Validation of adaptive threshold spikes detector for neural recording. Proceeding of IEEE EMBS, 2004). For the traces of neuronal activity in the STN and SN, in certain embodiments, it is the peak value within the duration, i.e., the single highest peak, for which a RMS is calculated and that value is used in the STN/SN ratio. The root mean square value of a function is often used in physics and electrical engineering. In the current invention, it is used to quantify neuronal firing of unit discharges. The pathophysiology of the Parkinson's Disease is the neuronal loss in the SN, and STN reactively becomes hyperactive. A simple, yet reliable way to quantify this pathophysiology is the STN/SN ratio of pRMS of neuronal activity. The pRMS may be determined as known to those of skill in the art.

In certain embodiments, a ratio as described herein may be determined using a meter, such as the FLUKE26III True RMS Multimeter, which is commercially available. Using the signals from the recording pass chosen for the final DBS lead implant, a ten second trace of neuronal firing from STN and a ten second trace from SN are input to the meter. For example, when the pRMS of the STN trace is 8 mV and the pRMS of the SN trace is 3 mV, then the ratio of STN/SN is 8/3=2.67.

In other certain embodiments, a ratio as described herein may be determined using a microprocessor 28, which is commercially available. Using the signals from the recording pass chosen for the final DBS lead 18 implant, a trace of neuronal firing from STN and a trace from SN are input to the microprocessor 28. For example, when the pRMS of the STN trace is 8 mV and the pRMS of the SN trace is 3 mV, then the ratio of STN/SN is 8/3=2.67. As further described elsewhere herein, such a ratio is indicative of the current status of the subject's Parkinson's Disease. A ratio value of 2.67 is indicative of advanced Parkinson's Disease. Note that a ratio determined according to this method is independent of impedance of the recording electrode since the two traces of signals were recorded by the same electrode (with the same Ω) at two different locations. It is suitable to compare such ratios across subjects. In other embodiments, a ratio as described herein may be determined using a Lead point brand micro-electrode recording system, which is commercially available as described elsewhere in this application. When using this system, a real time trace of neuronal firing from the STN results in the pRMS being automatically plotted against the depth to the target. The same may be accomplished for the SN. Then, the STN/SN ratio may be easily calculated. For example, when the pRMS of the STN trace is 6 mV and the pRMS of the SN trace is 5 mV, then the ratio of STN/SN is 6/5=1.2. As further described elsewhere herein, a ratio value of 1.2 is indicative of early Parkinson's Disease.

As described above, determination of the pRMS may be accomplished by a specific machine such as a volts meter FLUKE-26III True RMS Multimeter, or the Leadpoint advance analysis module, which is commercially available from Medtronic, Inc. of Minneapolis, Minn. 55423-5604. Upon completion of calculation of pRMS based upon the neuronal activity recorded in the STN and SN, a ratio of the pRMS of the STN to the pRMS of the SN may be calculated by using the pRMS for STN as the numerator and the pRMS of SN as the denominator.

As described above, determination of the pRMS may be accomplished by a specific machine such as the microprocessor 28. Upon completion of calculation of pRMS based upon the neuronal activity recorded in the STN and SN, a ratio of the pRMS of the STN to the pRMS of the SN may be calculated by using the pRMS for STN as the numerator and the pRMS of SN as the denominator. This determination may be made by the microprocessor 28. When the ratio is known, then the microprocessor 28 compares it to the information in FIG. 3 in order to determine the characteristics of the stimulation treatment to be communicated to the integrator 29 and ultimately the stimulator 12.

By way of background with regard to Parkinson's Disease, there is no known cause for the disease and diagnosis routinely revolves upon review and analysis of clinical symptoms. Definitive diagnosis of Parkinson's Disease may be accomplished by a post-mortum histological examination. Generally, diagnosis of Parkinson's Disease results from an investigation of clinical symptoms including tremors, rigidity, freezing, and dyskinesias. Rigidity is a symptom in which the muscles are too rigid to be moved. Dyskinesias is a symptom in which there is uncontrolled spontaneous movement. A summary of clinical symptoms is provided by the Unified Parkinson's Disease Reading Scale (UPDRS). The UPDRS is a number which is reflective of the status, or stage, of the Parkinson's Disease. For example, a UPDRS in the range of about 25 or less may be reflective of early Parkinson's Disease (or Hoehn and Yahr stage II) while a UPDRS in the range of about 40 or higher may be reflective of advanced Parkinson's Disease. By way of example, a ratio as calculated herein of around 1.5 may be reflective of early Parkinson's Disease. While a ratio of around 2.5 is reflective of advanced Parkinson's Disease. Obviously, the ratio is indicative of the status of Parkinson's Disease and as the status changes so should the characteristics of the electrical stimulation treatment.

The following Examples Section provides data and specific methods for obtaining neurological activity. As further described therein, the methods disclosed herein may be used to accurately and efficiently confirm a medical diagnosis of Parkinson's Disease. Given the high percentage of misdiagnosis of Parkinson's Disease, as well as the existence of several other medical conditions which mimic the clinical symptoms of Parkinson's Disease, there is a current and long lasting need for the inventions disclosed herein.

As known to those of ordinary skill in the art, there will be several sizes and different dimensions of the implantation lead 18, so that compatibility is provided for the many DBS insertion systems available from various manufacturers, such as CRW frame (Intergra, Inc.), Leksell Frame (Elekta Instruments, Inc), Nexfram (Medtronics) and StarFix (FHC, Inc.).

### Method of DBS

By way of background, the following procedure for implanting a brain stimulating macroelectrode, or lead, involves steps which generally include the following:
1. place a frame for stabilizing the insertion equipment on the subject,
2. perform imaging, according to a technique mentioned herein,
3. use the imaging data to ascertain the brain 22 target location,
4. drill an opening into the subject's head in order to access the subject's brain 22,
5. advance the implantable lead insertion assembly into the brain 22 until it is near the brain target location, as further described herein,
6. place the implantable lead at the exact brain target location, as further described herein,
7. take steps needed to test and then remove from the subject equipment used to place the implantable lead.

Other procedures for implanting a lead are known in the art, and include U.S. Pat. No. 7,450,997, entitled Method of Implanting a Lead For Brain Stimulation, which is hereby incorporated by reference herein. Methods for implanting items, such as a device 10 are known to those of skill in the art and are commonly practiced throughout the United States.

During a DBS implantable lead surgery, the rigid insertion assembly is inserted through a hole on the micropositioner aimed at the intended target, such as subthalamic nucleus. Referring now to FIG. 9, there is shown a schematic drawing of a side view of a brain 22. The approximate positions of the STN 64 and SN 66 are shown. Also shown is a lead 18 positioned to detect neuronal activity near the lead 18, for each. The connection of the lead 18 to the remainder of the equipment, as described herein, is not shown. By way of background, the coordinates for the location of STN are 12 mm lateral to the middle line of the brain, 4 mm posterior to the middle point of the AC-PC line (i.e., anterior commissure—posterior commissure line), and 4 mm below the AC-PC line. The SN is located 1 mm below the STN. Generally, the first end of the insertion lead is positioned about 10-28 mm from the brain target location so that the physiology of the target tissue is not disturbed by the intrusion. At this point, a recording microelectrode is inserted for physiological mapping. Physiological mapping is well known in the art. After the brain target location is determined physiologically, the next step is to switch the large DBS implantable lead with the mapping microelectrode.

Referring now to FIG. 10, there is shown a schematic drawing of a rear view of a brain 22. In certain embodiments, the procedure described herein occurs in a bilateral manner. Accordingly, shown is a dotted line which divides the left hemisphere of the brain 22 from the right hemisphere of the brain. A lead 18 may be inserted into an exact same location in each hemisphere so that neuronal activity of each hemisphere results. Also shown are the cables 32 which attach to a recording system 70. In certain embodiments, the device 10 disclosed herein may provide different outputs to different leads 18, or even different contacts on a lead 18, in order to provide appropriate treatment to each hemisphere of the brain 22 in a situation in which a plurality of leads 18 are implanted in a subject.

### EXAMPLES

### Example 1

### Obtaining an STN/SN Ratio

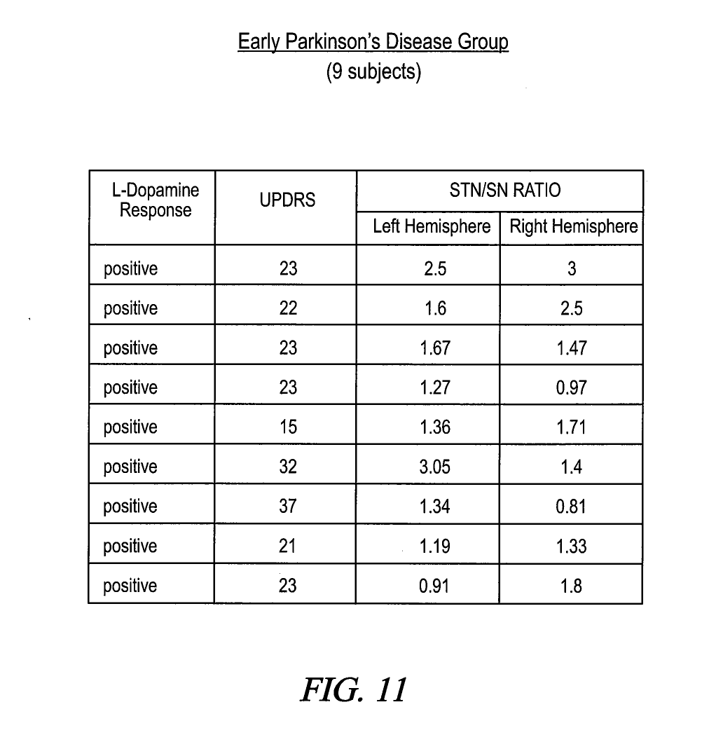

The following is an example of how STN/SN ratios were determined for eighteen age and sex matched subjects. The examples provide an understanding of the spectrum of STN/SN ratios that exist in subjects. The ages of the subjects range from 52 to 66 years old. The subjects were separated into an early Parkinson's Disease group and an advanced Parkinson's Disease group. Accordingly, the result was 9 subjects in the early Parkinson's Disease group and 9 subjects in the advanced Parkinson's Disease group. Figures relevant to these examples are FIGS. 11-14. The definition of a subject in the early Parkinson's Disease group is a subject that is 50 years old or older, that is within two years of the Parkinson's Disease diagnosis. The subjects in the advanced Parkinson's Disease group are medication-resistant following years of drug therapy, are 50 years old or older, and had an initial diagnosis of Parkinson's Disease 7-10 years ago. In order to obtain recordings of the activity of the subthalamic nucleus (STN) and the substantia nigra (SN), the procedure of multi-channel micro electrode recording (MER) was performed using a four channel Leadpoint brand micro-electrode recording system, which is commercially available from Medtronic, Inc., of Minneapolis, Minn. 55423-5604, or a ten channel Guideline 4,000 brand micro-electrode recording system, which is commercially available from FHC, Inc., of Bowdoin, Me. In certain embodiments, during a guided deep brain stimulation implant placement, a lead is inserted into the brain with 0.5 mm increments. During a bi-lateral symmetric procedure, a lead is placed into each hemisphere of the brain along identical paths with insertion occurring in identical increments. Neuronal activity is recorded at each 0.5 mm increment for a period of ten seconds. Accordingly, for example, there may be twelve ten second traces made while the lead traverses the subthalamic nucleus and twelve ten second traces made while the lead traverses the substantia nigra. Of the twelve traces, the trace having the highest peak is selected for the root mean square determinations disclosed herein. Thus, STN and SN recordings from the same tract of the final deep brain stimulation lead implants and the tract medial to the final implant were analyzed offline using the peak root mean square (pRMS) (Watkins P. T., Santhanam G., Shenoy K. V. and Harrison R. R., Validation of adaptive threshold spikes detector for neural recording. Proceeding of IEEE EMBS, 2004) using a volt meter available by the name FLUKE26III True RMS Multimeter to derive the STN/SN ratio. The ratio may also be derived from field potential recordings using a deep brain stimulation lead (Medtronic DBS3389) at the end of the implant surgery.

Because the procedure described above is a bilateral surgery, the results corresponding to the left hemisphere of the brain are noted by placing an L before STN. The ratios corresponding to the right hemisphere of the brain are noted as RSTN. Regarding the neurological activity recorded, an electrode on the lead which is present in the STN was used to record that activity. An element on the lead present within the SN, specifically within the substantia nigra pars compacta (SNc), was used to record the SN value.

According to several well known references, a sample size of 7 is sufficient to generate statistically relevant conclusions (Markowski C. A. and Markowski E. P., Conditions for the Effectiveness of a Preliminary Test of Variance; The American Statistician 44 (4): 322-326, 1990; Elise Whitley and Jonathan Ball, Statistics review 6: Nonparametric methods; Crit. Care 6(6): 509-513, 2002). As noted above, the sample size for each of the early and advanced groups is nine. Further, it is noteworthy that DBS procedures are extremely expensive such that the population undergoing them is limited. Also, there is no “control” group for the examples set forth herein. The minimal benefits of having a control group are significantly outweighed by the cost of the procedure and the risk associated with putting healthy individuals through the procedure in which leads are placed in the brain. Regarding the statistical relevance, the P values shown below were calculated according to commercially available software called Graph Pad software, which is commercially available from GraphPad Software, Inc., of San Diego, Calif. As noted in Example 3, the ratio was higher in the advanced Parkinson's Disease group compared to the early Parkinson's Disease group. Specifically, LSTN 2.59±0.72 for the advanced group as compared to the 1.65±0.69 for the early group; RSTN 2.49±0.67 for the advanced group as compared to 1.67±0.7 for the early group; the difference is highly significant with both p<0.05 unpaired t test, p<0.01 paired t test (Elise Whitley and Jonathan Ball, Statistics review 6: Nonparametric methods; Crit. Care 6(6): 509-513, 2002). It is noted that a non Parkinson's Disease subject has approximately equal activity in the STN and SN. Accordingly, the ratio for such a subject is around 1.

### Example 2

### Correlating the STN/SN Ratio to Known Information

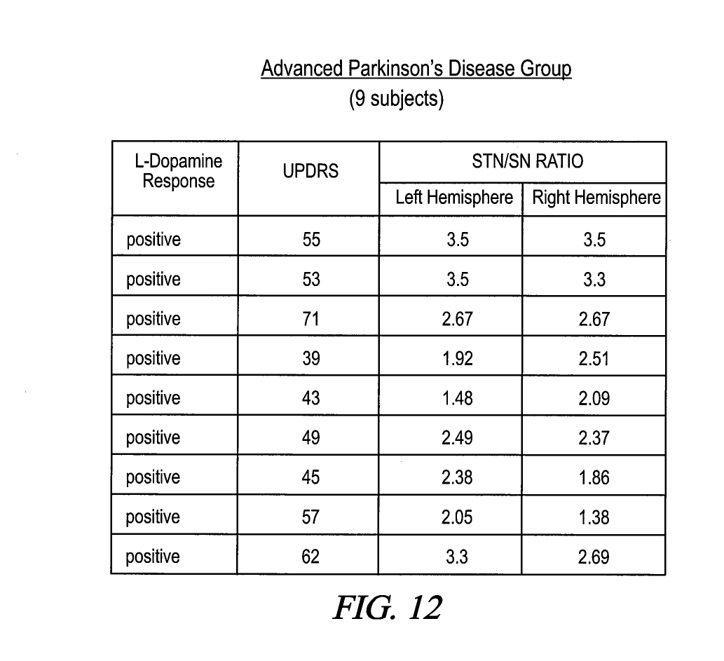

Referring now to FIGS. 11 and 12, there are shown the STN/SN ratios for the left hemisphere and right hemisphere along with the UPDRS values for each human subject. FIG. 11 shows the results for the early Parkinson's Disease group. FIG. 12 shows the results for the advanced Parkinson's Disease group. Those of skill in the art understand that the UPDRS values are considered the “gold standard” regarding the status of the subject's Parkinson's Disease. The Figures include additional information such as the subject's L-dopamine test results. The UPDRS values and ratios of each subject are shown.

### Example 3

### Utilization of the STN/SN Ratio

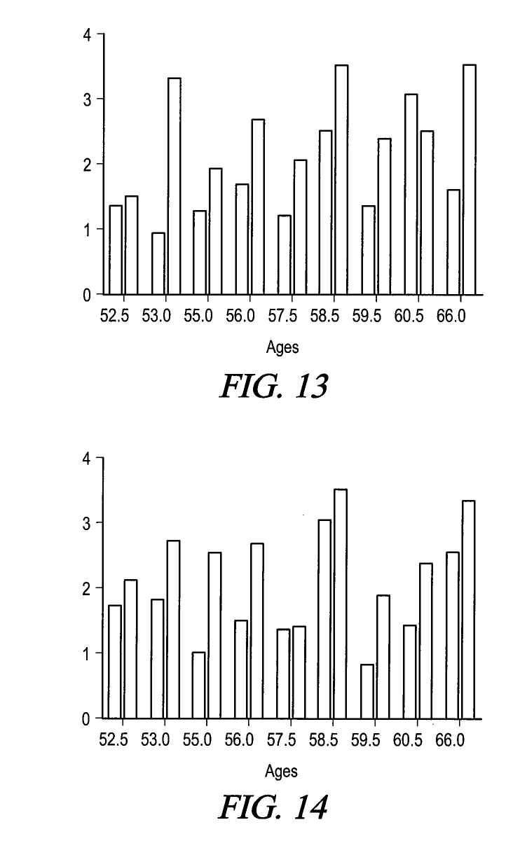

Review and analysis of the UPDRS values and the ratios reveals a relationship between the two which may be used to confirm a medical diagnosis of Parkinson's Disease. Confirmation of a medical diagnosis for Parkinson's Disease will prohibit a subject from undergoing medical procedures for that disease if the ratio does not confirm the existence of Parkinson's Disease. In the data provided herein, note that the higher ratio values correspond to the UPDRS values indicative of an advanced stage of Parkinson's Disease. Based upon the data provided, a STN/SN ratio of 2.5 or higher is indicative of advanced stage Parkinson's Disease. Referring now to FIGS. 13 and 14, there is shown the same data as displayed in FIGS. 11 and 12, however, it is shown in the form of bar graphs. Note that FIG. 13 shows the LSTN data and FIG. 14 shows the RSTN data. For each listed age, the bar graph on the left indicates the ratio for the early Parkinson's Disease subject and the bar graph on the right indicates the ratio for the advanced Parkinson's Disease subject. The ratios increase as the Parkinson's Disease advances. This is a further correlation that the STN/SN ratio is indicative of the severity of the Parkinson's Disease condition.

This patent application expressly incorporates by reference all patents, references, and publications disclosed herein.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1. A device for providing electrical stimulation, comprising:
a housing;
an electronic switch;
an amplifier attached to the electronic switch;
a converter attached to the amplifier;
a microprocessor attached to the converter and the housing;
an integrator attached to the microprocessor;
a stimulator attached to the integrator and the electronic switch.

2. The device of claim 1, further comprising a scanner operationally connected to the microprocessor.

3. The device of claim 2, further comprising a lead operationally connected to the electronic switch.

4. A recordation and stimulation system, comprising:
a lead;
a cable operationally connected to the lead;
a housing;
an electronic switch operationally connected to the cable;
an amplifier operationally connected to the electronic switch;
an analog to digital converter operationally connected to the amplifier, wherein the converter is attached to the housing;
a microprocessor operationally connected to the converter, wherein the microprocessor is attached to the housing;
an integrator operationally connected to the microprocessor;
a stimulator operationally connected to the integrator and the electronic switch, wherein the stimulator is attached to the housing.

5. The system of claim 4, further comprising a scanner operationally connected to the microprocessor.

6. A method of calculating a subthalamic nucleus to substantia nigra ratio, comprising:
measuring activity of a subthalamic nucleus;
measuring activity of a substantia nigra;
calculating a ratio of the subthalamic nucleus activity to the substantia nigra activity;
confirming a medical diagnosis of Parkinson's Disease based upon the ratio.

7. The method of claim 6, wherein confirming the medical diagnosis of Parkinson's Disease based upon the ratio further comprises comparing the ratio to a known ratio of a subject with early Parkinson's Disease or advanced Parkinson's Disease.

8. A method of obtaining a subthalamic nucleus to substantia nigra ratio, comprising:
inserting a lead into a brain;
recording a neuronal activity of an area of a subthalamic nucleus;
recording a neuronal activity of an area of a substantia nigra;
determining a ratio of the subthalamic nucleus neuronal activity to the substantia nigra neuronal activity;
displaying the ratio as a visual display.

9. The method of claim 8, further comprising comparing the ratio to at least one reference ratio comprising a ratio of neuronal activity in the subthalamic nucleus to neuronal activity in the substantia nigra for a subject having advanced Parkinson's Disease.

10. A method of determining the status of a Parkinson's Disease subject, comprising:
inserting a first lead into a brain;
inserting a second lead into the brain;
recording a neuronal activity of a subthalamic nucleus;
recording a neuronal activity of a substantia nigra;
determining a ratio of the neuronal activity of the subthalamic nucleus to the neuronal activity of the substantia nigra;
displaying the ratio as a visual display;
diagnosing Parkinson's Disease based upon the visual display.

11. The method of claim 10, wherein determining the ratio further comprises using a volts meter.

12. The method of claim 10, wherein diagnosing Parkinson's Disease further comprises comparing the ratio to a known ratio of a subject having Parkinson's Disease.

13. The method of claim 12, wherein the subject has early Parkinson's Disease.

14. The method of claim 12, wherein the subject has advanced Parkinson's Disease.
